(19) United States
(12) Patent Application Publication
(10) Pub. No.: US 2025/0218555 A1
(43) Pub. Date: Jul. 3, 2025

(54) Method and System for Recording Clinical Trial Data Using Generative AI Model

(71) Applicant: CRScube, Inc., Seoul (KR)

(72) Inventors: Seijun Kim, Seoul (KR); Gidae Yeo, Seoul (KR)

(21) Appl. No.: 18/776,848

(22) Filed: Jul. 18, 2024

(30) Foreign Application Priority Data
Dec. 29, 2023 (KR) 10-2023-0197437

(51) Int. Cl. G16H 10/20

(52) U.S. Cl. CPC G16H 10/20

(57) ABSTRACT

According to one aspect of the present invention, there is provided a method for recording clinical trial data, the method comprising the steps of: acquiring clinical trial data included in an area specified by a user; generating structured data using the clinical trial data; and recording key-value data included in the structured data as data on an item corresponding to the key.

### FIELD OF THE INVENTION

The present invention relates to a method and system for recording clinical trial data.

### BACKGROUND

A variety of data collected through clinical trials are inputted into an electronic data capture (EDC) system to be analyzed and studied. Because clinical trials are conducted in a hospital, the data collected through clinical trials is also basically inputted into an electronic medical record (EMR) system, and a substantial part of such data should be inputted into the EDC system.

However, it is difficult for the EMR system and the EDC system to interwork due to privacy and other reasons, so that a clinical research coordinator (CRC) or other person in charge in the hospital visually identifies the data inputted into the EMR system and manually inputs the identified data into the EDC system. Further, human errors such as typos may occur during the input process, which may cause significant inefficiencies such as a clinical research associate (CRA) visiting the hospital and separately performing a verification procedure called source data verification (SDV).

In this connection, the inventor(s) present a technique to assist in remedying inefficiencies that occur during the process of collecting clinical trial data.

### SUMMARY OF THE INVENTION

One object of the present invention is to solve all the above-described problems in prior art.

Another object of the invention is to acquire clinical trial data included in an area specified by a user, generate structured data using the clinical trial data, and record key-value data included in the structured data as data on an item corresponding to the key.

Yet another object of the invention is to enable data recorded in a first system (e.g., EMR system) to be easily recorded in a second system (e.g., EDC system).

The representative configurations of the invention to achieve the above objects are described below.

According to one aspect of the invention, there is provided a method comprising the steps of: acquiring clinical trial data included in an area specified by a user; generating structured data using the clinical trial data; and recording key-value data included in the structured data as data on an item corresponding to the key.

According to another aspect of the invention, there is provided a system comprising: a clinical data acquisition unit configured to acquire clinical trial data included in an area specified by a user; a structured data generation unit configured to generate structured data using the clinical trial data; and a clinical data recording unit configured to record key-value data included in the structured data as data on an item corresponding to the key.

In addition, there are further provided other methods and systems to implement the invention, as well as non-transitory computer-readable recording media having stored thereon computer programs for executing the methods.

According to the invention, it is possible to acquire clinical trial data included in an area specified by a user, generate structured data using the clinical trial data, and record key-value data included in the structured data as data on an item corresponding to the key.

According to the invention, it is possible to enable data recorded in a first system (e.g., EMR system) to be easily recorded in a second system (e.g., EDC system).

### BRIEF DESCRIPTION OF THE DRAWINGS

FIG. 1 schematically shows the configuration of an entire system for recording clinical trial data according to one embodiment of the invention.

FIG. 2 specifically shows the internal configuration of a clinical trial data management system according to one embodiment of the invention.

FIG. 3 illustratively shows how to acquire unstructured data according to one embodiment of the invention.

FIG. 4 illustratively shows how to generate structured data according to one embodiment of the invention.

FIG. 5 illustratively shows how an area including clinical trial data is specified by a user according to one embodiment of the invention.

FIG. 6 illustratively shows how to acquire the clinical trial data included in the area described with reference to FIG. 5.

FIG. 7 illustratively shows how to generate structured data according to one embodiment of the invention.

FIG. 8 illustratively shows how to extract key-value data from structured data according to one embodiment of the invention.

FIG. 9 illustratively shows how to extract key-value data from structured data according to one embodiment of the invention.

FIG. 10 illustratively shows how to map a key and a corresponding item according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures, and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

### Configuration of the Entire System

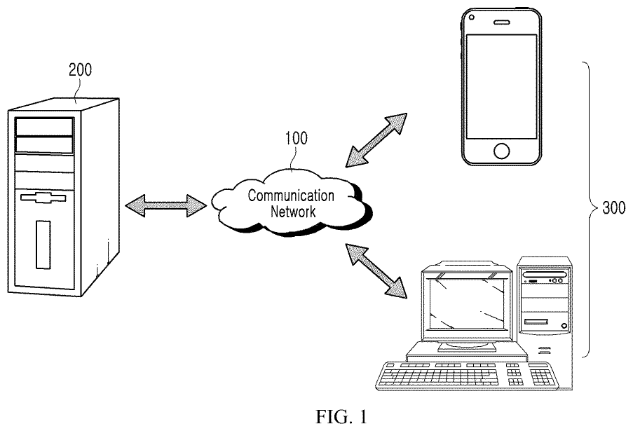

FIG. 1 schematically shows the configuration of the entire system for recording clinical trial data according to one embodiment of the invention.

As shown in FIG. 1, the entire system according to one embodiment of the invention may comprise a communication network 100, a clinical trial data management system 200, and a device 300.

First, the communication network 100 according to one embodiment of the invention may be implemented regardless of such as wired and wireless communication modality communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, 5G communication, Bluetooth communication (including Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Next, the clinical trial data management system 200 according to one embodiment of the invention may function to acquire clinical trial data included in an area specified by a user, generate structured data using the clinical trial data, and record key-value data included in the structured data as data on an item corresponding to the key.

The configuration and functions of the clinical trial data management system 200 according to the invention will be discussed in more detail below.

Next, the device 300 according to one embodiment of the invention is digital equipment capable of connecting to and then communicating with the clinical trial data management system 200, and any type of digital equipment having a memory means and a microprocessor for computing capabilities, such as a smart phone, a tablet, a smart watch, a smart band, smart glasses, a desktop computer, a notebook computer, a workstation, a personal digital assistant (PDAs), a web pad, and a mobile phone, may be adopted as the device 300 according to the invention.

In particular, the device 300 may include an application (not shown) for assisting a user to receive services such as clinical trial data management from the clinical trial data management system 200. The application may be downloaded from the clinical trial data management system 200 or an external application distribution server (not shown). Meanwhile, the characteristics of the application may be generally similar to those of a clinical data acquisition unit 210, a structured data generation unit 220, a clinical data recording unit 230, a communication unit 240, and a control unit 250 of the clinical trial data management system 200 to be described below. Here, at least a part of the application may be replaced with a hardware device or a firmware device that may perform a substantially equal or equivalent function, as necessary.

### Configuration of the Clinical Trial Data Management System

Hereinafter, the internal configuration of the clinical trial data management system 200 crucial for implementing the invention and the functions of the respective components thereof will be discussed.

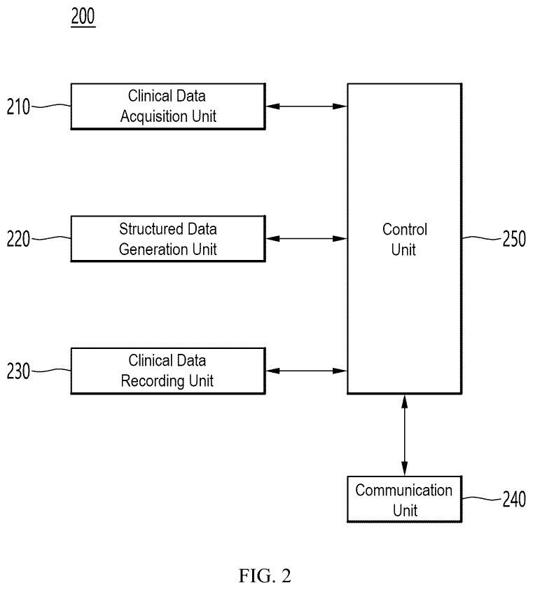

FIG. 2 specifically shows the internal configuration of the clinical trial data management system 200 according to one embodiment of the invention.

As shown in FIG. 2, the clinical trial data management system 200 according to one embodiment of the invention may comprise a clinical data acquisition unit 210, a structured data generation unit 220, a clinical data recording unit 230, a communication unit 240, and a control unit 250. According to one embodiment of the invention, at least some of the clinical data acquisition unit 210, the structured data generation unit 220, the clinical data recording unit 230, the communication unit 240, and the control unit 250 may be program modules to communicate with an external system (not shown). The program modules may be included in the clinical trial data management system 200 in the form of operating systems, application program modules, or other program modules, while they may be physically stored in a variety of commonly known storage devices. Further, the program modules may also be stored in a remote storage device that may communicate with the clinical trial data management system 200. Meanwhile, such program modules may include, but are not limited to, routines, subroutines, programs, objects, components, data structures, and the like for performing specific tasks or executing specific abstract data types as will be described below in accordance with the invention.

Meanwhile, the above description is illustrative although the clinical trial data management system 200 has been described as above, and it will be apparent to those skilled in the art that at least a part of the components or functions of the clinical trial data management system 200 may be implemented in the device 300 or a server (not shown) or included in an external system (not shown), as necessary.

For example, according to one embodiment of the invention, all of the components and functions of the clinical trial data management system 200 may be implemented in the device 300, so that the user may receive services according to embodiments of the invention in an on-device manner. As another example, according to one embodiment of the invention, all of the functions of the clinical trial data management system 200, the EMR system, and/or the EDC system may be implemented in the device 300 via a web browser or the like.

First, the clinical data acquisition unit 210 according to one embodiment of the invention may function to acquire clinical trial data included in an area specified by a user. Specifically, according to one embodiment of the invention, an area including clinical trial data may be specified by the user. The area may be specified in a variety of manners. Although some embodiments thereof will be described below, the manners are not limited to the described embodiments and may be diversely changed as long as the objects of the invention may be achieved. Of course, clinical trial data acquired by the clinical data acquisition unit 210 according to one embodiment of the invention may be included in at least one area specified in a plurality of manners.

For example, according to one embodiment of the invention, the area including clinical trial data may be specified by the user photographing (e.g., in a camera preview state) an area including clinical trial data (e.g., at least a part of a screen shown on a display of the device 300) using a user device (not shown) such as a smart phone or a tablet. Here, the user device may be a separate device distinct from the device 300 described with reference to FIG. 1. Further, the clinical data acquisition unit 210 according to one embodiment of the invention may perform optical character recognition (OCR) on the specified area to acquire clinical trial data included in the area. According to one embodiment of the invention, a tool such as Google ML Kit may be used for the OCR.

Here, according to one embodiment of the invention, a camera guide for the OCR may be provided to the user to increase the accuracy of the OCR. For example, the user may be provided with a camera angle guide using a gyro sensor. As another example, the user may be provided with a guide based on information on light around the photographing location using a light sensor, an image sensor, or the like. As another example, according to one embodiment of the invention, the area including clinical trial data may be specified by the user designating at least a part of an area shown on the display of the device 300 that includes clinical trial data (e.g., dragging an arbitrary area or dragging an arbitrary range of text) via a mouse/keyboard input, a touch input, or the like. More specifically, when the clinical trial data included in the specified area is copied to a clipboard via a user input, the clinical data acquisition unit 210 according to one embodiment of the invention may acquire the copied data as the clinical trial data included in the specified area.

As another example, according to one embodiment of the invention, the clinical data acquisition unit 210 according to one embodiment of the invention may perform OCR on the area specified by the user's designation as above to acquire the clinical trial data included in the area. According to one embodiment of the invention, the OCR in this case may refer to OCR for a browser (e.g., Chrome) or other framework (e.g., a standalone EMR system). Further, a tool (or program module) in the form of an extension (or plugin) may be used for the OCR.

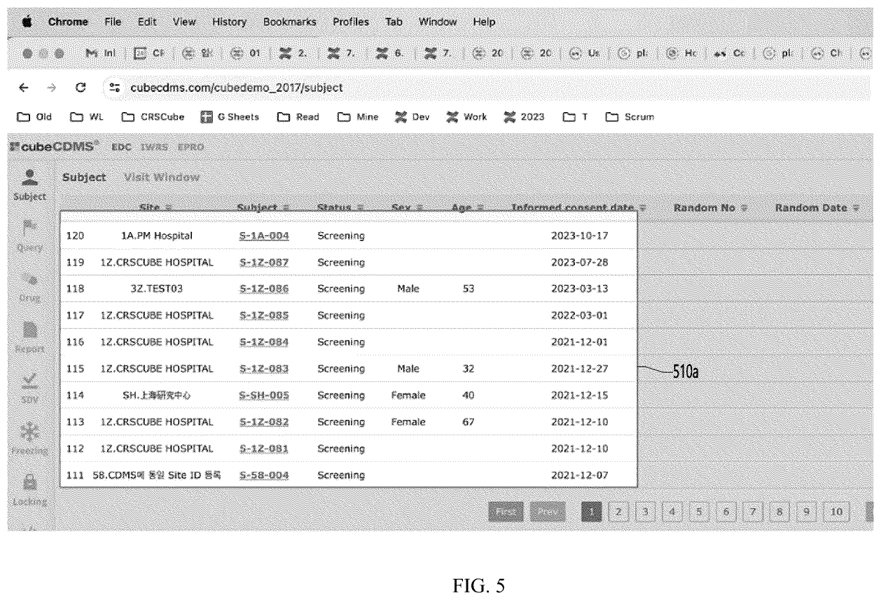

FIG. 5 illustratively shows how an area including clinical trial data is specified by a user according to one embodiment of the invention.

Referring to FIG. 5, when an area 510a including clinical trial data is specified by the user designating the area 510a, the clinical data acquisition unit 210 according to one embodiment of the invention may acquire the clinical trial data by performing OCR on the specified area 510a. Meanwhile, in acquiring the clinical trial data in various manners as described above, the clinical data acquisition unit 210 according to one embodiment of the invention may remove personal information such as a name or chart number of a patient (or clinical trial subject) for privacy protection, or the personal information may be removed before the clinical trial data is acquired.

Next, the structured data generation unit 220 according to one embodiment of the invention may function to generate structured data using the clinical trial data acquired by the clinical data acquisition unit 210.

Specifically, according to one embodiment of the invention, the clinical trial data acquired by the clinical data acquisition unit 210 may be unstructured data. According to one embodiment of the invention, the unstructured data may retain the structural features of source data (or original data) to some extent, but may also be completely (or nearly) devoid of the structural features.

For example, when the source data is in a tabular format, the clinical trial data acquired by the clinical data acquisition unit 210 according to one embodiment of the invention may retain the row and/or column structure of the source data to some extent, or may have the structure of a one-dimensional list completely (or nearly) devoid of the row and column structure.

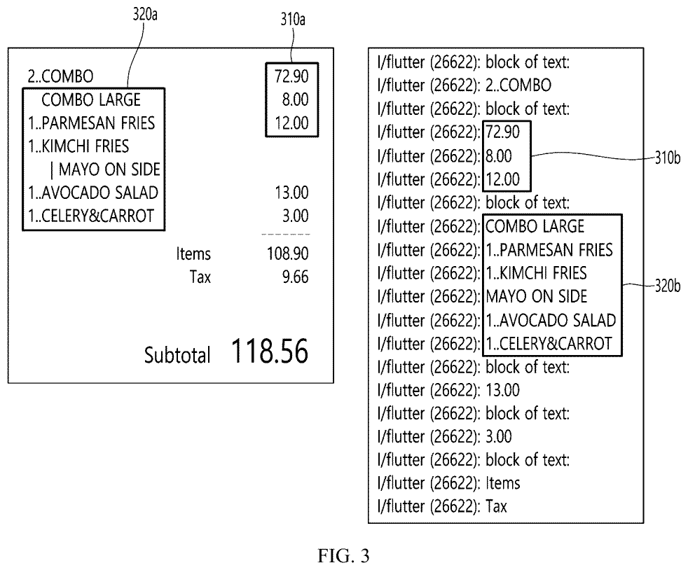

FIG. 3 illustratively shows unstructured data according to one embodiment of the invention.

In order to facilitate understanding of the invention, FIG. 3 shows an example of unstructured data acquired by performing OCR on a receipt that has been or is being photographed, although the acquired data is not clinical trial data. As shown in FIG. 3, text in specific areas 310a and 320a included in the image may be recognized as the OCR is performed, and the recognized text may be divided into separate blocks 310b and 320b. The data in the block 310b and the data in the block 320b are unstructured data that are simply textual and do not correlate with each other (e.g., although the text “COMBO LARGE” and the text “72.90” are recognized, it is not possible to tell from the OCR results alone whether the price of the COMBO LARGE is 72.90).

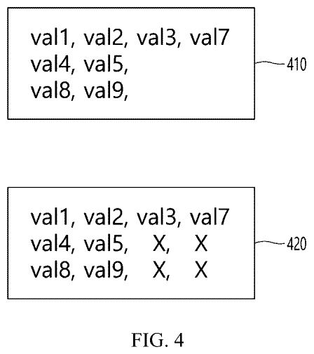

FIG. 4 illustratively shows how to generate structured data according to one embodiment of the invention.

As described above, the unstructured data acquired by the clinical data acquisition unit 210 according to one embodiment of the invention may retain the structural features of source data to some extent. As shown in FIG. 4, unstructured data 410 may retain row information of source data in a tabular format. Meanwhile, four values missing in the unstructured data 410 may have been lost due to problems in the data acquisition process (e.g., errors in the OCR process), or may not have been present in the source data in the first place. The process of generating the structured data will be described later.

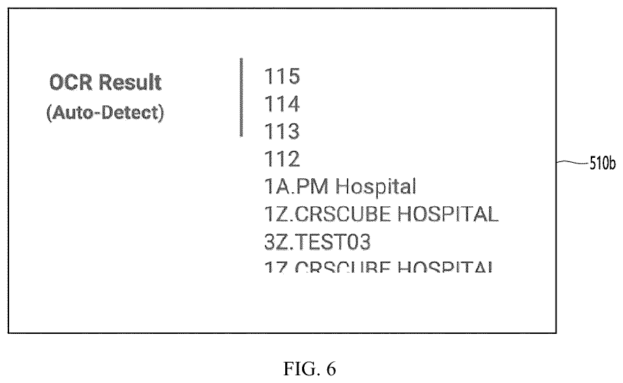

FIG. 6 illustratively shows how to acquire the clinical trial data included in the area described with reference to FIG. 5.

Referring to FIGS. 5 and 6, OCR may be performed on an area 510a specified by the user to acquire clinical trial data 510b included in the area 510a (only a part of the data to be acquired is shown). As shown, the data 510b may be unstructured data 510b having the structure of a one-dimensional list (i.e., the structure of [“115”, “114”, “113”, “112”, “1A.PM Hospital”, . . . ]) completely devoid of the structural features (e.g., row and column structure) of the source data.

Further, the structured data generation unit 220 according to one embodiment of the invention may function to generate structured data using the clinical trial data acquired by the clinical data acquisition unit 210. Here, the clinical trial data may be the unstructured data acquired as described above.

For example, referring again to FIG. 4, the structured data generation unit 220 according to one embodiment of the invention may generate structured data 420 by processing the unstructured data 410 and converting it into a tabular format. As shown in FIG. 4, the structured data 420 may be generated by filling predetermined values (denoted by X) in where data is absent. According to one embodiment of the invention, a generative AI model may be utilized for the conversion. Here, the generative AI model according to one embodiment of the invention may be generated by fine-tuning a publicly available generative AI model such as ChatGPT using appropriate prompts.

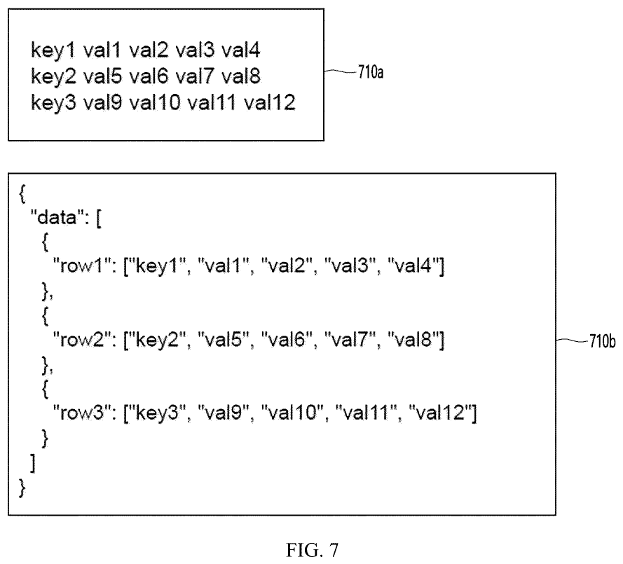

FIG. 7 illustratively shows how to generate structured data according to one embodiment of the invention.

Referring to FIG. 7, as another example, structured data 710a generated by the structured data generation unit 220 to one embodiment of the invention converting unstructured data into a tabular format may be converted again into structured data 710b in a JSON (JavaScript Object Notation) format. According to one embodiment of the invention, a generative AI model may be utilized to generate the structured data in the JSON format. Here, the generative AI model according to one embodiment of the invention may be generated by fine-tuning a publicly available generative AI model such as ChatGPT using appropriate prompts.

Meanwhile, FIG. 7 shows the structured data 710a and 710b such that the data in the first column of each row are treated as keys and the data in the other columns of each row are treated as values. However, this is only for convenience of illustration, and it should be understood that the position of the key in each row may vary depending on the nature of the data. Further, in some cases, at least a part of the values shown in FIG. 7 (i.e, val1 to val12) may correspond to the X described above (see 420 in FIG. 4).

The structured data generation unit 220 according to one embodiment of the invention may also convert the unstructured data into a tabular format on the basis of patterns in the unstructured data.

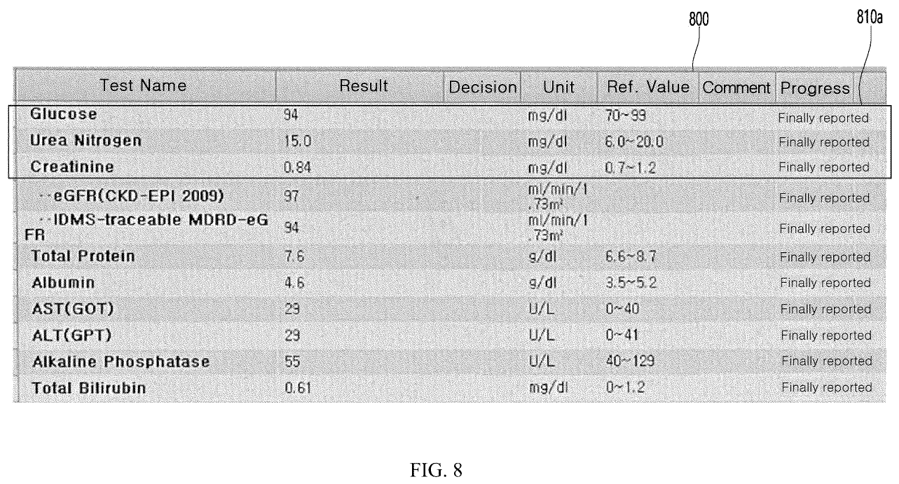

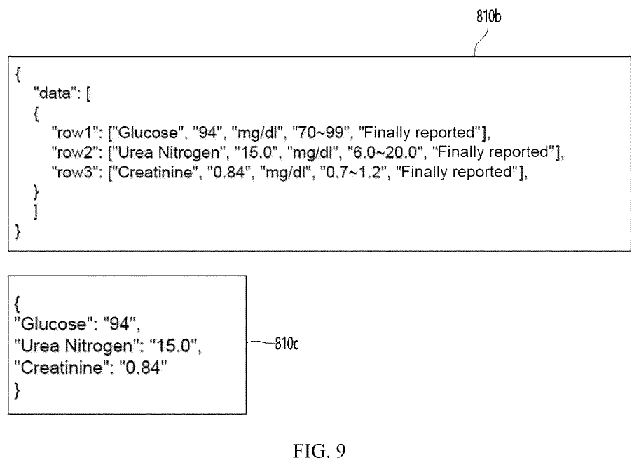

FIGS. 8 and 9 illustratively show how to extract key-value data from structured data according to one embodiment of the invention.

Referring to FIG. 8, for example, the clinical data acquisition unit 210 according to one embodiment of the invention may perform OCR on an area 810a specified by the user in an area 800 shown in a window of an EMR system, thereby acquiring clinical trial data included in the area 810a. As described above, the acquired data may be unstructured data having the structure of a one-dimensional list (e.g., the structure of [“Glucose”, “94”, “mg/dl”, “70-99”, “Finally reported”, “Urea Nitrogen”, “15.0”, “mg/dl”, “6.0-20.0”, “Finally reported”, “Creatinine”, . . . ]) completely devoid of the structural features of the source data 810a.

Further, the unstructured data having the structure of a one-dimensional list may require rows to be distinguished in order to be converted into a tabular format, which may be achieved by using a generative AI model. Here, the generative AI model according to one embodiment of the invention may be generated by fine-tuning a publicly available generative AI model such as ChatGPT using appropriate prompts. In this case, the prompts may be designed to distinguish rows on the basis of patterns in the unstructured data (e.g., while sequentially traversing the list, treat each occurrence of specific text as a new row, or if the same format of data occurs at intervals of specific addresses (or addresses of list elements), distinguish rows on the basis of the address intervals). Referring to FIG. 9, the structured data generation unit 220 according to one embodiment of the invention may generate structured data 810b in the aforementioned JSON format with the rows being distinguished as above.

Next, when the structured data is generated by the structured data generation unit 220, the clinical data recording unit 230 according to one embodiment of the invention may function to record key-value data included in the structured data as data on an item corresponding to the key.

Specifically, the clinical data recording unit 230 according to one embodiment of the invention may extract key-value data from the structured data and record the extracted key-value data as data on an item corresponding to the key. Here, according to one embodiment of the invention, the key-value data may include one key and at least one value.

Referring again to FIGS. 8 and 9, for example, the clinical data recording unit 230 according to one embodiment of the invention may extract key-value data 810c from the structured data 810b and record the extracted key-value data 810c as data on an item corresponding to the key. More specifically, the clinical data recording unit 230 according to one embodiment of the invention may treat the data in the first column (i.e., the column corresponding to “Test Name” in 800) of each row of the structured data 810b as a key, and treat the data in the second column (i.e., the column corresponding to “Result” in 800) of each row as a value, and may extract the data in those columns as the key-value data 810c. Meanwhile, the above description is only provided for illustrative purposes to facilitate understanding, and it is of course possible to treat data in a different column of each row as a key, or treat data in more than one column as values.

Further, the clinical data recording unit 230 according to one embodiment of the invention may cause a key (e.g., “Glucose”) and a value (e.g., “94”) associated therewith, i.e., key-value data, to be recorded in an EDC system as data on an item corresponding to the key (e.g., “Glucose” or a predetermined item mapped thereto), i.e., may cause the same data as the key-value data to be inputted to a corresponding item in the EDC system.

Meanwhile, the clinical data recording unit 230 according to one embodiment of the invention may utilize a generative AT model to extract the key-value data from the structured data as described above. Here, the generative AI model according to one embodiment of the invention may be generated by fine-tuning a publicly available generative AI model such as ChatGPT using appropriate prompts.

Here, the clinical data recording unit 230 according to one embodiment of the invention may map the key and the corresponding item so that the key-value data extracted as above is recorded as data on the corresponding item.

In general, data inputted into the EMR system, i.e., data acquired by the clinical data acquisition unit 210 according to one embodiment of the invention, are often treated as different types of data due to differences in notation and the like, even if they are related to substantially the same key. Thus, by mapping the key and the corresponding item as above, data related to substantially the same key may be treated as the same type of data. This may assist in more accurate analysis of clinical trial data. The clinical data recording unit 230 according to one embodiment of the invention may utilize a generative AI model to map the key and the corresponding item as described above. Here, the generative AI model according to one embodiment of the invention may be generated by fine-tuning a publicly available generative AI model such as ChatGPT using appropriate prompts.

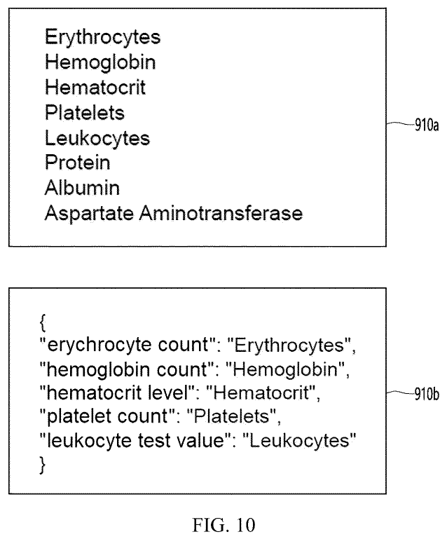

FIG. 10 illustratively shows how to map a key and a corresponding item according to one embodiment of the invention.

Referring to FIG. 10, corresponding items 910a may be pre-inputted values. The clinical data recording unit 230 according to one embodiment of the invention may map keys (“erythrocyte count”, “hemoglobin count”, “hematocrit level”, “platelet count”, and “leukocyte test value”) to corresponding items using a generative AI model, respectively, and output the results as data 910b in a JSON format so that the key-value data are recorded as data on the corresponding items.

However, the keys and the corresponding items according to one embodiment of the invention are not limited to those listed above, and may be diversely changed as long as the objects of the invention may be achieved.

Next, the communication unit 240 according to one embodiment of the invention may function to enable data transmission/reception from/to the clinical data acquisition unit 210, the structured data generation unit 220, and the clinical data recording unit 230.

Lastly, the control unit 250 according to one embodiment of the invention may function to control data flow among the clinical data acquisition unit 210, the structured data generation unit 220, the clinical data recording unit 230, and the communication unit 240. That is, the control unit 250 according to one embodiment of the invention may control data flow into/out of the clinical trial data management system 200 or data flow among the respective components of the clinical trial data management system 200, such that the clinical data acquisition unit 210, the structured data generation unit 220, the clinical data recording unit 230, and the communication unit 240 may carry out their particular functions, respectively.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for recording clinical trial data, the method comprising the steps of:
acquiring clinical trial data included in an area specified by a user;
generating structured data using the clinical trial data; and
recording key-value data included in the structured data as data on an item corresponding to the key.

2. The method of claim 1, wherein in the acquiring step, the area is specified by being photographed by the user with a camera, and the clinical trial data is acquired by performing optical character recognition (OCR) on the area.

3. The method of claim 2, wherein in the acquiring step, a camera guide for the OCR is provided to the user.

4. The method of claim 1, wherein in the acquiring step, the area is specified by being designated by the user.

5. The method of claim 4, wherein in the acquiring step, the clinical trial data is acquired by performing OCR on the area.

6. The method of claim 1, wherein the clinical trial data is unstructured data.

7. The method of claim 6, wherein in the generating step, the structured data is generated by converting the unstructured data into a tabular format.

8. The method of claim 7, wherein in the generating step, the unstructured data is converted into the tabular format on the basis of patterns in the unstructured data.

9. The method of claim 1, wherein the recording step comprises the step of extracting the key-value data from the structured data, and the key-value data includes one key and at least one value.

10. The method of claim 1, wherein in the recording step, the key-value data is recorded as the data on the corresponding item by mapping the key and the corresponding item.

11. A non-transitory computer-readable recording medium having stored thereon a computer program for executing the method of claim 1.

12. A system for recording clinical trial data, the system comprising:
a clinical data acquisition unit configured to acquire clinical trial data included in an area specified by a user;
a structured data generation unit configured to generate structured data using the clinical trial data; and
a clinical data recording unit configured to record key-value data included in the structured data as data on an item corresponding to the key.

13. The system of claim 12, wherein the area is specified by being photographed by the user with a camera, and the clinical data acquisition unit is configured to acquire the clinical trial data by performing optical character recognition (OCR) on the area.

14. The system of claim 13, wherein a camera guide for the OCR is provided to the user.

15. The system of claim 12, wherein the area is specified by being designated by the user.

16. The system of claim 15, wherein the clinical data acquisition unit is configured to acquire the clinical trial data by performing OCR on the area.

17. The system of claim 12, wherein the clinical trial data is unstructured data.

18. The system of claim 17, wherein the structured data generation unit is configured to generate the structured data by converting the unstructured data into a tabular format.

19. The system of claim 18, wherein the structured data generation unit is configured to convert the unstructured data into the tabular format on the basis of patterns in the unstructured data.

20. The system of claim 12, wherein the clinical data recording unit is configured to extract the key-value data from the structured data, and the key-value data includes one key and at least one value.

21. The system of claim 12, wherein the clinical data recording unit is configured to record the key-value data as the data on the corresponding item by mapping the key and the corresponding item.
